(19) United States
(12) Patent Application Publication
(10) Pub. No.: US 2009/0069629 A1
(43) Pub. Date: Mar. 12, 2009

(54) SEXUAL PLEASURE STIMULATION DEVICE

(75) Inventor: Aurelio Uson Calvo, Madrid (ES)

Correspondence Address: RICHARD M. GOLDBERG, 25 EAST SALEM STREET, SUITE 419, HACKENSACK, NJ, 07601, US

(73) Assignees: Aurelio Uson Calvo, Madrid (ES); INVENCIONES MEDICO SANITARIAS, Madrid (ES)

(21) Appl. No.: 11/577,607

(22) PCT Filed: Jul. 26, 2005
(86) PCT No.: PCT/ES05/00420

§ 371 Date: Apr. 20, 2007

(30) Foreign Application Priority Data
Oct. 29, 2004 (ES) P200402622

(51) Int. Cl. A61F 5/00; A61H 1/00

(52) U.S. Cl. 600/38; 601/46

(57) ABSTRACT

The invention relates to an external penile prosthesis or device which is designed to stimulate a couple's sexual pleasure during vaginal intercourse. The inventive device has been designed ergonomically on anatomical/physiological bases that are related to the erogenous zones of the male and female genitals. The device consists of a duckbill-shaped head, three ventral rings and a posterior appendage. According to the invention, each of said components performs a particular, specific function. The device is used by the man during vaginal intercourse after it has been duly positioned on the external genitals, such that the above-mentioned head lies longitudinally against the dorsal face of the erect penis where it is fixed in place by the distal, central and proximal rings. The proximal ring is placed around the root of the penis, behind the scrotal sac, such that the semi-spherical posterior appendage remains in contact with the perianal skin of the user.

### AIM OF THE INVENTION

This invention relates to the patent of invention application corresponding to a new external and casual penile prosthesis or device, the main aim of which is to stimulate and/or heighten a couple's sexual pleasure, during vaginal intercourse. This unique device has been devised, designed and shaped ergonomically on the basis of anatomical fundamentals and physiological knowledge associated with the so-called erogenous zones, in both the male and female genitals.

Given its ergonomic shape, as will be described later, this new device, once duly positioned on the man's external genitals, with its duckbill-shaped head lying longitudinally against the dorsal face of the proximal end of the penis, will, during vaginal intercourse, gradually and progressively stimulate the erogenous areas of the woman's genitals, in other words; the clitoris and the “G” and “H” points, in the front third of the upper wall of the vagina.

This new external ergonomic penile prosthesis or device is made up of three basic components, interrelated to achieve the main aim, which is: “To allow the man and woman to experience maximum sexual pleasure during vaginal intercourse”.

### FIELD OF THE INVENTION

In the light of the above, it is easy to deduce that the main field of practical application of the new erogenous device that the invention proposes, is those heterosexual couples that want to improve and/or further enjoy vaginal intercourse and, particularly, in those men or women whose intimate relationship as man and wife, or a couple, has deteriorated due to the sexual dissatisfaction of one of the two parties. In this respect, the disagreements and/or confrontations which, with certain frequency, question the quality of sexual relationships in the lives of married and/or common-law couples, are common knowledge, alleging the woman's sexual dissatisfaction during vaginal intercourse, either due to behavioural or psycho/sexological changes in her partner, or due to anatomical, functional and/or pathological causes in both the man and woman's genitals.

Therefore, the new sexual pleasure stimulation device, during vaginal intercourse, that is proposed by the invention, straightaway has a worldwide client base of unusual proportions, because the immense majority of men and women who are single or living together as a married couple, and heterosexual common-law couples would like to mutually experience maximum sexual pleasure during vaginal intercourse, ending the dissatisfaction that generally occurs frequently to women during vaginal intercourse.

For its part, this new external penile prosthesis or device used by the man, to satisfy the woman during intercourse, has another field of application within the industry that specialises in manufacturing external prostheses, complements, accessories and/or sanitary products.

### BACKGROUND TO THE INVENTION

At this point, it is interesting to recall that Dr. D. Aurelio V. Usón Calvo is the owner of a utility model, Publication No. 1047478 and application No.: 200001681, relating to a “Sexual pleasure stimulation device”, granted by the SPTO on 1 Sep. 2001, indicating here and now that said device has become obsolete, with some structural changes being made to the current model, which have led to significant improvements for users, including, for example, the following:

1. The new device is shaped ergonomically as one single piece, which makes it easier for the user to put it on and remove it more comfortably, more quickly and more safely.

2. The new device includes one extra ring in its configuration, located in the middle of the ventral area of the head, which increases the head's support on the dorsal face of the penis.

3. The proximal ring in the new device includes a much wider nipple than its predecessor, in the concavity of the top arch and, also, a larger undercut, in the rear-upper wall of said ring.

4. Finally, the proximal ring of the new device is now an integral, elastic piece that can adapt around the root of the scrotal sac and, on the rear face of the lower arch, it incorporates the so-called posterior appendix or appendage.

In view of the above, neither in the sanitary market nor in specialised medical literature nor in the catalogues of products offered by sex shops, have we been able to find any other sexual pleasure stimulation device, during vaginal intercourse, that can compare with the external penile prosthesis proposed by the invention. Furthermore, none of the so-called devices currently used by men to maintain and/or heighten their erection during vaginal intercourse, can compare with the structure, ergonomic shape and efficiency characterising the new device that the invention introduces. This is all due to the fact that the new sexual pleasure stimulation device has been devised, designed and shaped ergonomically, on the basis of certain anatomical-physiological fundamentals, associated with the so-called erogenous areas, in the external genitals of both sexes.

Finally, given its softness, malleability and adaptability to the morphology of external male and female genitals, no significant user discomfort or complications are envisaged, particularly, if said device is positioned correctly and is used according to the “instructions recommended for using the device correctly”, which are included in each unit of this novel sexual pleasure stimulation device. Also, in some cases, it will be necessary to apply a lubrication gel to the head of the stimulation device, particularly for users who have a large penis or for women with atrophic vaginitis, little or no vaginal lubrication, or other pathological causes and/or congenital malformations.

### DESCRIPTION OF THE INVENTION

The new sexual pleasure stimulation device that the invention proposes, is shaped ergonomically and made up of three basic components which, in turn, are interrelated so that, easily and without any significant problems or complications, users can achieve the purpose sought by the invention, in other words: “to mutually experience and, if possible, to the same degree, maximum sexual pleasure during vaginal intercourse”. There are three main components making up this new and unique sexual pleasure stimulation device:

1. The duckbill-shaped head. This first component, once positioned and adjusted along the dorsal face of the proximal end of the erect penis, will, during vaginal intercourse, provide a wider and more erect penis. Also, the “humpback element” or projection in the middle third of the head's dorsal face will, during vaginal intercourse, stimulate the erogenous areas of the woman's genitals, heightening her sexual pleasure and/or enjoyment which, in turn, will be transmitted to her partner and shared by him.

2. The three rings, distal, central and proximal. The particular and, in turn, shared function of these three rings is to firmly attach the head at its base along the dorsal face of the proximal end of the erect penis, for the duration of the sexual intercourse.

The proximal ring is the largest and widest of the three and it is firmly attached to the head by the front face of the top arch. This proximal ring is also intended to compress the deep dorsal vein in the penis, thanks to the nipple or projection incorporated in the concavity of the top arch. Said nipple will delay the spontaneous and/or premature appearance of the backflow of venous blood during vaginal intercourse and, therefore, the erection will be maintained for longer. On the other hand, if said backflow does occur, as happens fairly frequently during the sexual act, the erection will disappear prematurely. Furthermore, when this proximal ring is positioned behind the root of the scrotal sac, it will make said sac and also its contents (the testicles) project forwards and upwards. Therefore, the user will achieve a good intermittent contact with the skin of the woman's perineal and vulvar area during vaginal intercourse, which, generally speaking, does not occur in normal situations.

The two remaining rings, that is: the central and distal rings reinforce the attachment of the head in the centre and at its distal end, respectively.

3. The posterior appendage or tentacle. This third component of the sexual pleasure stimulation device is made up of a piece like an ‘upturned spoon’ with one end joined to the rear face of the lower arch of the proximal ring, and having at the other end a semi-spherical part, which remains in contact with the user's perineal and/or perianal skin. During vaginal intercourse, this semi-spherical part will gently and repeatedly stimulate the above-mentioned areas of skin, delaying the appearance of the orgasmic and/or ejaculatory backflow, among other reasons because the vaginal intercourse will be prolonged for longer.

The material used to manufacture this sexual pleasure stimulation device, during vaginal intercourse, can be medical grade silicon or latex or any other of the synthetic products approved by the Ministry of Health for the manufacture of external sanitary prostheses.

Each and every one of the components making up the new sexual pleasure stimulation device, can be modified or varied in terms of their structure, morphological shape, size and product consistency. Also new additions can be added or the smooth surface of the head can be modified, making it rough, grooved, with downiness and even bristled. Furthermore, the duckbill-shaped head can include a ball or roller within the “humpback part” element that will slide gently, rubbing the clitoris or when passing underneath the vulvar-vaginal fork, and/or rubbing the mucous on the top-front face of the vaginal wall. Instead of comprising a fixed, non-removable humpback element, the device can include a humpback element that extends as the user wishes and even including a vibration action of variable intensity, which can be activated or deactivated and, also, regulated, by an emitter controlled by the users.

Finally, the external sexual pleasure stimulation device, during vaginal intercourse, proposed by the invention can be used on its own, as described in this specification, or on top of or around the penis previously sheathed in a normal condom and, even, backing on to a mini condom inserted into the distal ring of said stimulator, which will only cover the gland and its coronary groove, leaving the rest of the penis uncovered, albeit bearing the device as already explained. This way, an important element is added to the new sexual pleasure stimulation device, particularly, for those users who do not want to have children.

### DESCRIPTION OF THE DRAWINGS

To complete the description of the invention and to increase comprehension of the morphological and functional characteristics thereof, a sheet of drawings accompanies this specification, and is an integral part thereof, which are a non-limiting, illustrative representation, as follows:

FIGS. 1, 2, 3 and 4. Represent four drawings or views of the new Sexual Pleasure Stimulation Device, from four angles or different perspectives. These 4 drawings have been prepared on the computer, and give a detailed and accurate image of the situation and ergonomic design of the three components making up and shaping said device: A) The head, B) The rings; (1) the proximal ring, (2) the central ring, (3) the distal ring, and C) The posterior appendage.

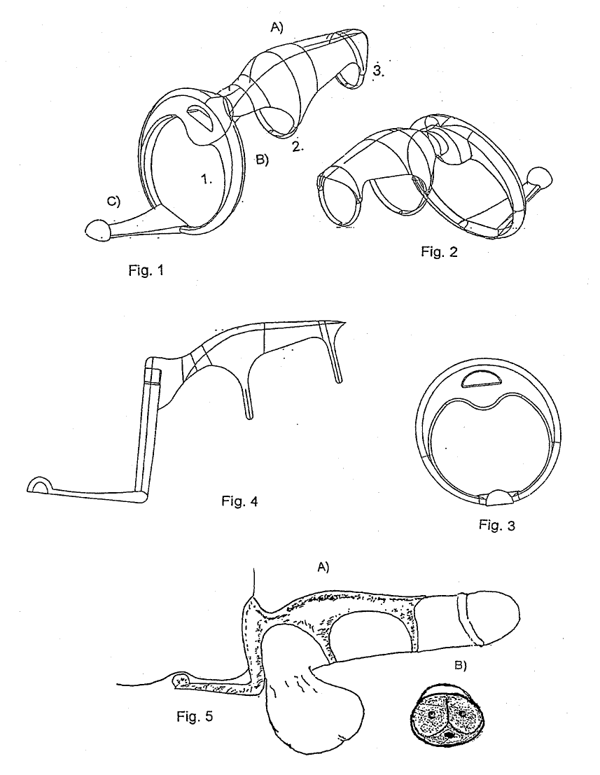

In these drawings it is possible to appreciate the location and design of each and every one of the said components making up the device, as well as their interrelation, so that it is easier to achieve the intended aim during vaginal intercourse. Note C) the posterior appendage shaped like an “upturned spoon”, attached to the rear face of the lower arch of the proximal ring and finishing at the other end in a semi-spherical part that projects upwards to gently and repeatedly tap the user's perianal and perineal skin during vaginal intercourse, delaying the appearance of the ejaculatory reflux. See FIG. 5.

FIG. 5. This consists of two elevation drawings, showing A) a side perspective of the erect penis bearing the new sexual pleasure stimulation device, before starting vaginal intercourse. Note the increased thickness of the proximal end of the penis, the upper profile of the dorsal face of the head bulging in the shape of a duck's bill forming a “humpback element” or projection in its proximal third. Also, see the proximal ring positioned around the root of the penis and the scrotal sac, projecting it forward and upwards, as well as the testicles. The figure also shows the position and shape of the central and distal rings. Finally, it shows the semi-spherical part at the distal end of the posterior appendage, in contact with the skin in the perianal or perineal area which part, during vaginal intercourse, with a gentle, repetitive and intermittent tap, partially prevents the so-called orgasmic and/or ejaculatory reflux, thus prolonging the duration of the vaginal intercourse. Drawing B) shows a front view of a transverse section of the erect penis which also includes the “humpback element” of the sexual pleasure stimulation device.

### PREFERRED EMBODIMENT OF THE INVENTION

In view of the descriptive information provided in this specification and, also, complemented by the attached sheet of drawings, it can be deduced that the new sexual pleasure stimulation device for couples, during vaginal intercourse, proposed by the invention, is a welcome event, expected for a long time and which, in our opinion, will contribute to the well being of men and women in the 21^{st} century, improving the quality of their sexual relations that are felt and shared mutually during vaginal intercourse.

In the light of the above, it is not considered necessary to provide a further description so that an expert and, even, any normal adult, can understand the importance and the scope of the invention, as well as the advantages it provides. As for the type and/or variety of materials used, and the consistency, malleability, shape, size and number of constituent elements, these can be modified in any way, providing, it does not imply altering the essence of the invention.

What is claimed is:

1. Device for stimulating the sexual pleasure of a man and woman, during vaginal intercourse, comprising a duckbill-shaped head having a humpback element, intended to stimulate, through repeated contact, erogenous areas of female genitals during vaginal intercourse owing to the humpback element provided on said head, said humpback element lying longitudinal to a dorsal face of a proximal third of an erect penis when positioned thereon.

2. Device for stimulating the sexual pleasure of a man and woman couple, during vaginal intercourse, according to claim 1, further comprising three rings in a ventral part thereof, intended to attach said head at a base thereof lying longitudinally to a top face of a proximal end of the erect penis.

3. Device for stimulating the sexual pleasure of a man and woman, during vaginal intercourse, according to claim 2, wherein one of the three rings is a proximal ring that is much larger and wider than the other two rings and, in turn, has a nipple in a concavity of an upper arch thereof that will be used to compress a deep dorsal vein of the penis, thus preventing detumescence or premature slackening of the erection and, therefore, prolonging vaginal intercourse for a longer period of time.

4. Device for stimulating the sexual pleasure of a man and woman, during vaginal intercourse, according to claim 3, wherein the device projects the scrotal sac and contents thereof forwards and upwards, owing to the positioning behind said scrotal sac of the proximal ring, which will produce gentle contact with skin in a woman's vulvar-vaginal and front perineal area, helping to heighten the couple's mutual sexual pleasure, during vaginal intercourse.

5. Device for stimulating the sexual pleasure for a man and woman, during vaginal intercourse, according to claim 2, wherein includes a posterior appendix that emerges from a rear face of a lower arch of the proximal ring, said appendix ending in a semi-spherical part seated on a top surface of said appendix and having its sole and exclusive function being, during vaginal intercourse, to repeatedly stimulate skin in the perineal and perianal area of a user delaying, in this way, so-called organism or ejaculatory reflux.
